(19) United States
(12) Patent Application Publication
(10) Pub. No.: US 2003/0014092 A1
(43) Pub. Date: Jan. 16, 2003

(54) Apparatus for the treatment of glaucoma

(76) Inventor: Thomas Neuhann, Muenchen (DE)

Correspondence Address: JORDAN AND HAMBURG LLP, 122 EAST 42ND STREET, SUITE 4000, NEW YORK, NY, 10168, US

(21) Appl. No.: 10/090,974

(22) Filed: Mar. 5, 2002

(30) Foreign Application Priority Data
Apr. 18, 2001 (DE) 101 18 933.8-51

(51) Int. Cl. A61N 1/00

(52) U.S. Cl. 607/89

(57) ABSTRACT

An apparatus for the treatment of glaucoma with a laser catheter and a light-conducting fiber arrangement, to the proximal end of which light can be coupled and at the distal end of which a light-emerging surface is provided, with a pressure-sensitive device to achieve a specified contracting pressure of the light-emerging surface.

The inventory relates to an apparatus for the treatment of glaucoma with a laser catheter and a light-conducting fiber arrangement, to the end of which light can be coupled and at the distal end of which a light-emerging surface is provided.

There are medicinal procedures as well as a series of surgical procedures for treating glaucoma and eliminating excess pressure in the eyeball. Moreover, an apparatus of the type described above has also already been proposed. For this apparatus, the spongy trabecula mechanism, through which he aqueous humor flows from the front and the rear chambers of the eye, is removed locally by means of UV light, which is produced preferably by an excimer laser, so that the aqueous humor can more easily reach the Schlemm canal, through which it is finally discharged.

For using this apparatus, it is necessary to open the eye locally, in order to pass the light with the help of tale light-conducting laser catheter into the immediate vicinity of the tissue of the trabecula mechanism, which is to be perforated. For this purpose, it is necessary to position the light-emerging surface precisely in front of the Schlemm canal, in order to perforate the trabecula mechanism precisely at this place and to ensure the discharge into the Schlemm canal. However, this positioning of the distal end of the laser catheter is very difficult and, as a result, makes the use of such an apparatus for the treatment of glaucoma to a certain degree a matter of luck, for which it is important to actually find the correct position when introducing the laser catheter.

A further, important problem when such an apparatus is used for the treatment of glaucoma with a laser catheter consists of maintaining a specified contacting pressure, so that the trabecula mechanism is not pierced mechanically by the light-conducting fiber arrangement and this light-conducting fiber arrangement can also not slip away laterally from the desired contacting position.

It is therefore an object of the invention to configure an apparatus of the type named above so that the positioning o the distal end of the light-conducting fiber arrangement is guaranteed, while a specified contacting pressure is ensured.

This objective is accomplished by an apparatus of the type named above for the treatment of glaucoma, wherein a pressure-sensitive device is provided for achieving a specified contacting pressure of the light-emerging surface.

Such a pressure-sensitive device may be an elastic component, which can be slipped onto the distal end and has a contacting lip, which can be bent. This contacting lip may also be configured so that it has a supportive function in the chamber angle and, with that, prevents the fiber arrangement from slipping from the previously placed position.

In the case of such a contacting device with contacting lips, which can be bent, the contacting of the tip is signaled to the surgeon not only by a reaction force, which results from the force required for the bending, but also is indicated to him by the bending of the contacting lips, which can be seen in the microscope. Accordingly, the surgeon can determine the correct position by observing the bent contacting lip.

In a further development of the invention, provisions can be made so that the laser catheter, in the region outside of the eye, has two sections, which can be shifted mutually against a spring arrangement. Preferably, an optical and/or acoustic display device maybe provided to indicate that a specified contacting pressure has been reached or exceeded.

In a further development of the invention, an overload safety device may also be provided, which, when a specifiable contacting pressure of the light-emerging surface of the light-guiding fiber arrangement has been exceeded, interrupts the fictional connection of the sections, so that an accidental piercing is reliably prevented.

The pressure-sensitive apparatus may also be constructed from non-elastic components (employing, for example, a membrane or crystal technique) on the assumption that it is ensured that the user perceives the pressure attained. Preferably, an optical and/or acoustic display device is provided to signal that a specified contacting pressure has been attained or exceeded.

Finally, it is also within the scope of the invention, that the distal end of the light-guiding fiber arrangement is provided with a device, which preferably can be slipped op and prevents the light-emerging surface slipping off, this device having an arbor or being formed owing to the fact that the slip-on element consists of an anti-slip material, to which the trabecular mechanism adheres.

Further advantages, distinguishing features and details of the invention arise out of the following description of an example and from the drawing in which

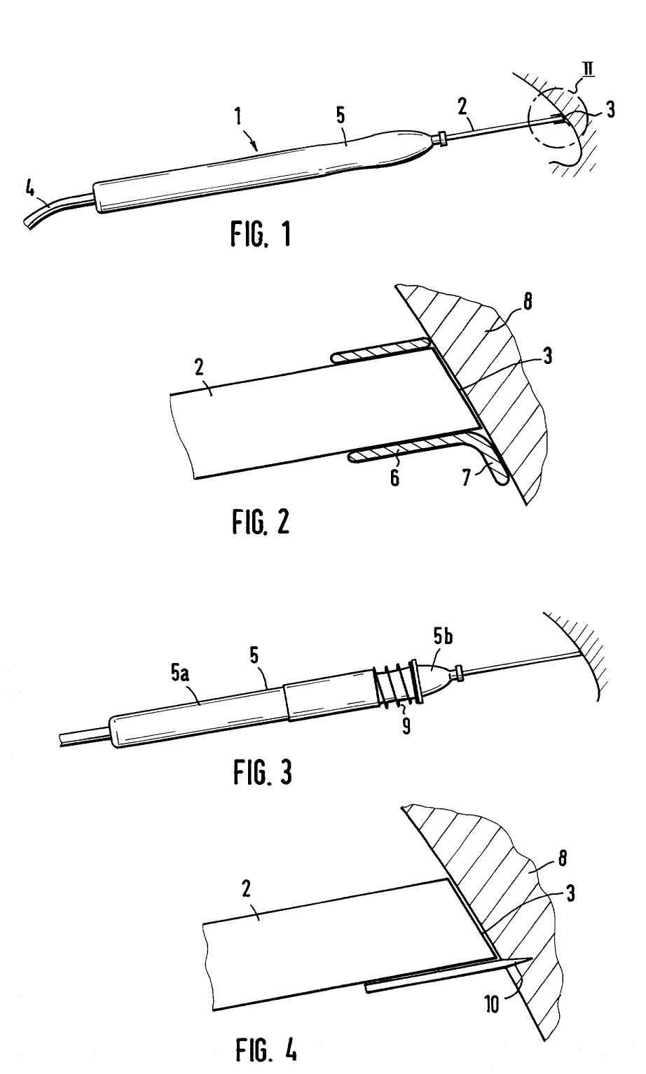

FIG. 1 shows a diagrammatic representation of an inceptive apparatus with a contacting lip, which can be bent,

FIG. 2 shows al enlarged sectional representation of the region II in FIG. 1,

FIG. 3 shows a diagrammatic representation of a second embodiment of an inventive apparatus with elastic sections, which can be shifted axially with respect to one another and

FIG. 4 shows an enlarged sectional representation of the distal end of the light-conducting fiber arrangement of a further embodiment of an inventive glaucoma treatment apparatus with a slippage-preventing device in the form of an arbor.

The glaucoma treatment apparatus, shown diagrammatically in FIG. 1, comprises a laser catheter 1 with a light-guiding fiber arrangement 2 at the distal end of which a light-emerging surface 3 is provided. The light is supplied by a laser source, which is not shown, over a fiber-optical light guide 4. The handle 5 of the laser catheter, with the help of which the light-conducting fiber arrangement 2 with the light-emerging surface can be positioned in the eye under the microscopic supervision by the surgeon, is also shown diagrammatically.

In the case of the example shown in FIGS. 1 and 2, a sleeve 6 with a contacting lip 7, which can be bent, is slipped onto the distal end of the light-conducting fiber arrangement 2, the contacting lip 7, in the state of rest, being inclined obliquely to the outside with respect to the axis of the fiber arrangement, so that it can be bent to the outside only, as shown in FIG. 2, when pressed against the trabecula mechanism. Not only can a specified contacting pressure be set or obtained by this contacting lip 7, but the surgeon, due to the bending of this contacting lip, can also recognize that the light-emerging surface 3 has come to lie against the trabecula mechanism 8 and can then switch on the laser source.

In the case of the embodiment shown in FIG. 3, which may also, in addition, comprise the contacting apparatus with the contacting lip 7 of FIGS. 1 and 2, the laser catheter is divided into two parts in the region of the handle 5, it being possible to shift the parts 5a and 5b axially with respect to one another against the force of a spring 9. To form a contact of the lip of the light-conducting fiber arrangement 2 with the danger of piercing the tissue can be avoided by adjusting the force of the spring 9. If necessary, an overload protection may be provided, which is not shown and is initiated only when a specified contacting pressure is exceeded, releasing the parts 5a and 5b, so that they can then be shifted freely with respect to one another.

An optical all acoustic display device for signaling that a specified contacting pressure has been exceeded, in that either a light-emitting diode lights up or an acoustic signal sounds, which calls the attention of the surgeon to the fact that the laser catheter must not be moved forwards further to prevent a pressure being exceeded, is also not shown.

A variation of the embodiment in FIG. 3 could be a device, which is realized with non-elastic components (employing, for example, a membrane or crystal technique), which are also pressure-sensitive.

Finally, FIG. 4 diagrammatically shows the distal end of the light-conducting fiber arrangement 2 with an arbor 10, which is glued to the side and protrudes at the front and secures a position by piercing the trabecula mechanism 8, so that a lateral slipping of the light-emerging surface 3 and, with that, a change in the position in front of the Schlemm canal lying in the trabecula mechanism, is prevented.

The arbor may, of course, also be fastened to a sleeve-shaped component, which can be slipped onto the tip of the light-conducting fiber arrangement 2.

The invention is not limited to the example shown. In particular, it would also be possible to slip a component, with or without the contacting lip that can be bent as in the example of FIGS. 1 and 2, onto the distal end of the light-conducting fiber arrangement 2, which consists of an anti-slip material. Such a material could, for example, be one with a particular microstructure, which can prevent such slipping off.

1. An apparatus for the treatment of glaucoma with a laser catheter and a light-conducting fiber arrangement, to the proximal end of which light can be coupled and at the distal end of which a light-emerging surface is provided, wherein a pressure-sensitive device (6, 7, 9) is provided to achieve a specified contacting pressure of the light-emerging surface (3).

2. The apparatus of claim 1, wherein the distal end has an elastic component (6) with a contacting lip (7), which can be bent.

3. The apparatus of claim 2, wherein the contacting lip (7) is constructed so that it is supportive in the chamber angle and, with that, prevents the fiber arrangement slipping off at the previously-placed site.

4. The apparatus of one of the claims 1 to 3, wherein the laser catheter (1), in the region outside of the eye, has two sections (5a, 5b), which can be shifted axially with respect to one another against a spring arrangement (9).

5. The apparatus of one of the claims 1 to 3 wherein the laser catheter (1), in the region outside of the eye, has a pressure-sensitive device, which is realized with non-elastic components (employing, for example, a membrane or crystal technique), which are also pressure sensitive.

6. The apparatus of claim 4, wherein an overload safety device interrupts the frictional connection of the sections (5a, 5b) when a specifiable contacting pressure of the light-emerging surface (3) of the light-conducting fiber arrangement (2) is exceeded.

7. The apparatus of one of the claims 4 to 6, wherein an optical display device and/or an acoustic indicator signal that a specified contacting pressure has been attained or exceeded.

8. An apparatus, especially for treating glaucoma with a laser catheter and a light-conducting fiber arrangement, into the proximal end of which light can be coupled and at the distal end of which a light-emerging surface is provided, wherein the distal end of the light-conducting fiber arrangement is provided with a device, which preferably can be slipped on and prevents the contacting light-emerging surface slipping off.

9. The apparatus of claim 8, wherein the device, which prevents slipping off has an arbor (10).

10. The device of claim 8, wherein the device, which prevents slipping off, consists of an anti-slip material, which adheres to the trabecula mechanism.
